(19) United States
(12) Patent Application Publication
(10) Pub. No.: US 2025/0129391 A1
(43) Pub. Date: Apr. 24, 2025

(54) SYSTEM AND METHOD FOR CONTROLLED POLYMER DEPOLYMERIZATION

(71) Applicant: Intropic Materials Corporation, Emeryville, CA (US)

(72) Inventors: Aaron Hall, Oakland, CA (US); Adam Safir, Oakland, CA (US); Jennifer Imbrogno, Oakland, CA (US); Jolene Mattson, Oakland, CA (US); Rodolfo Orellana, Oakland, CA (US); Stefan Marco Eres, Oakland, CA (US)

(73) Assignee: Intropic Materials Corporation, Emeryville, CA (US)

(21) Appl. No.: 18/920,640

(22) Filed: Oct. 18, 2024

Related U.S. Application Data
(60) Provisional application No. 63/592,000, filed on Oct. 20, 2023.

(51) Int. Cl. C12P 1/00; C08K 5/00

(52) U.S. Cl. CPC C12P 1/00; C08K 5/0033

(57) ABSTRACT

A system can include a polymer degradant and a nano-protectant. The nano-protectant preferably surrounds (e.g., encloses, encapsulates, etc.) the polymer degradant. The system can optionally be incorporated into a polymeric substance. A method for forming a polymeric nano-protectant can include forming a mixture from a plurality of distinct monomers and polymerizing the plurality of distinct monomers.

### CROSS-REFERENCE TO RELATED APPLICATIONS

This application claims the benefit of U.S. Provisional Application No. 63/592,000 filed 20 Oct. 2023, which is incorporated in its entirety by this reference.

### TECHNICAL FIELD

This invention relates generally to the polymer disposal field, and more specifically to a new and useful system and method in the polymer disposal field.

### BRIEF DESCRIPTION OF THE FIGURES

FIG. 1 is a schematic representation of an exemplary embodiment of the invention.

FIGS. 2A-2D are chemical drawings of exemplary monomers for use in a polymeric nano-protectant. In these examples, monomers that include more than one R group, the R groups can be the same or different. In these examples, the R groups can include alkanes (e.g., branched or linear alkanes with between 0-20 carbon atoms), cycloalkanes (e.g., with between 3 and 20 carbon atoms, inclusive of carbon atoms bonded to the ring), polycyclicalkanes (e.g., spiro compounds, bridging compounds, fused rings, etc. with more than one ring each containing between 0-20 carbon atoms), aromatics (e.g., with between 0-20 carbon atoms and 0-10 heteroatoms such as nitrogen, oxygen, phosphorous, sulfur, etc. in the aromatic ring), ions (e.g., alkali metal cations, alkaline earth metal cations, ammonium, etc.), hydrogen, and/or any suitable R group(s) can be used.

FIGS. 3A-3D are schematic representations of exemplary clusters of enzymes (e.g., a first enzyme E1 and/or a second enzyme E2) embedded in exemplary nanoprotectants (e.g., a first nanoprotectant NP1 and/or a second nanoprotectant NP2).

FIGS. 4A-4B are schematic representations of exemplary nanoprotectant-enzyme complexes where the nanoprotectant is designed to interact with an active site of an enzyme or an exposed surface area of an enzyme respectively.

FIG. 5 is a schematic representation of an exemplary polymeric nanoprotectant.

FIG. 6 is a schematic representation of an example of radical polymerization used to form polymer nanoprotectants.

### DETAILED DESCRIPTION

The following description of the embodiments of the invention is not intended to limit the invention to these embodiments, but rather to enable any person skilled in the art to make and use this invention.

### 1. Overview

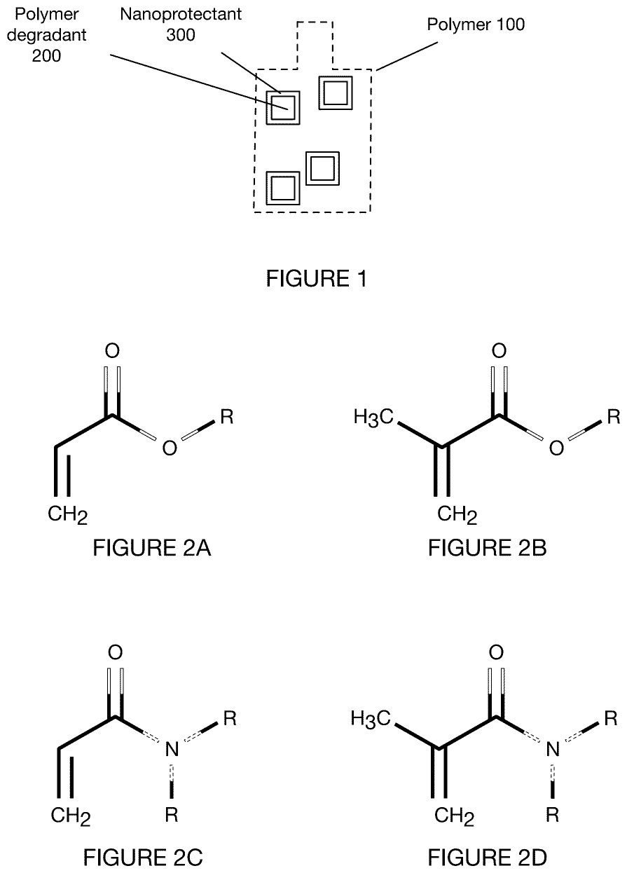

As shown in FIG. 1, a composition (e.g., a polymer degrading complex, polymer degrading composition, etc.) can include a polymer degradant and a nano-protectant. The nano-protectant preferably surrounds (e.g., encloses, encapsulates, etc.) the polymer degradant. However, the nano-protectant and polymer degradant can otherwise be related. The composition (e.g., a polymer degrading complex, polymer degrading composition, etc.) can optionally be incorporated into a polymeric substance.

The composition (e.g., a polymer degrading complex, polymer degrading composition, etc.) preferably functions to degrade (e.g., depolymerize) a polymeric substance (where a polymeric substance can include oligomers, polymers, combinations of polymers and oligomers, and/or other suitable species generated from polymerization style reactions between monomers). For instance, the polymer degradant can be selected to degrade a target polymeric substance (polymeric resin). The degradation of the polymeric substance is preferably a sequential degradation (e.g., processive depolymerization, progressive depolymerization, etc.). For example, the polymeric substance can be degraded monomer by monomer (e.g., removing one monomer from an end of the polymeric substance at a time). However, the polymeric substance could be degraded oligomerically (e.g., removing a dimer, trimer, tetramer, or other oligomer from the polymer until the polymer is full broken down), chemically (e.g., oxidize the polymer into carbon dioxide, water, and other trace species such as nitrogen oxides, sulfur oxides, etc.), via chain cleavage (e.g., breaking the polymeric substances at positions along the backbone that differ from locations where monomers directly reacted in the formation of the polymeric substance), by random chain scission (typically but not necessarily between monomeric linkage sites of the polymeric substance), and/or can be otherwise degraded. In some variants, the monomers (and/or oligomers) resulting from degradation of the polymer can be recycled (e.g., used to make new polymeric substance). However, the composition (e.g., a polymer degrading complex, polymer degrading composition, etc.) can otherwise function (e.g., degrade or breakdown a polymeric substance in any manner).

In a specific example, the composition (e.g., a polymer degrading complex, polymer degrading composition, etc.) can include a PETase enzyme (e.g., as a polymer degradant) enclosed within a nano-protectant (e.g., a random or pseudorandom heteropolymer such as a statistically distributed polymer formed from methyl methacrylate (MMA), 2-ethylhexyl methacrylate (EHMA), 3-sulfopropyl methacrylate (SPMA), and oligo (ethylene glycol) methyl ether methacrylate (OEGMA). In this specific example, the composition can be incorporated into a PET polymer (e.g., shopping bag, bottle, containers, carpet, clothes, packaging films, etc.), where after a threshold condition (e.g., time, temperature, force, chemical exposure, etc.), the PETase can depolymerize the PET.

### 2. Technical Advantages

Variants of the technology can confer one or more advantages over conventional technologies.

First, variants of the technology can mitigate (e.g., minimize, hinder, reduce, remove, etc.) the formation of microplastics from the breakdown of plastic waste and/or reduce the amount of plastic that is discarded. For example, inclusion of a plastic degradant that degrades the plastic into monomers (e.g., via processive depolymerization) in the plastic can reduce (and potentially prevent) the formation of microplastics and/or discarding the plastic (e.g., because the monomers can be reused to form plastic, can be converted to other chemicals for further chemical processes, etc.).

Second, variants of the technology can enable (e.g., facilitate) potentially unstable (e.g., unstable to polymer manufacturing, unstable to incorporation into the polymer, unstable to classes of solvents, unstable to ultraviolet light, etc.) polymer degradants (e.g., plastic degradants) to be incorporated into a polymer. For example, the use of a nano-protectant can protect the polymer degradant from decomposing during exposure to conditions that the polymer degradant is susceptible to and can facilitate the incorporation of the polymer degradant into a polymer (e.g., without significantly impacting the structural, functional, etc. properties of the polymer).

However, further advantages can be provided by the composition and method disclosed herein.

### 3. Composition

As shown in FIG. 1, a composition (e.g., a polymer degrading complex, polymer degrading composition, etc.) can include a polymer degradant and a nano-protectant. The nano-protectant preferably surrounds (e.g., encloses, encapsulates, etc.) the polymer degradant. However, the nano-protectant and polymer degradant can otherwise be related. The composition can optionally be incorporated into a polymeric substance.

The polymeric substance is typically a plastic (e.g., thermoplastic, thermosetting polymer, fibre-reinforced plastic, corrugated plastic, polymeric foam, high-performance plastic, etc.). However, the polymeric substance can additionally or alternatively include an elastomer (e.g., rubber), an adhesive (e.g., binders, sealants, epoxy, glue, etc.), a biopolymer (e.g., polysaccharide, natural rubber, suberin, lignin, polyhydroxyalkanoates, cutin, cutan, melanin, starch, cellulose, RNA, DNA, proteins, etc.), and/or can include any suitable polymeric material(s). The polymeric substance can be a commodity plastic, engineering plastic, and/or high-performance plastic.

The polymeric substance can be or include fibers, resins, extruded shapes (e.g., tubes, rods, etc.), foam, coatings, adhesives, potting, sealant, and/or in any suitable form.

The polymeric substance can be crystalline, semi-crystalline, nano-crystalline, amorphous, and/or can have any suitable crystallinity.

A number average molecular weight (e.g., measured based on colligative properties such as freezing point depression, end group analysis, 1H NMR, gel permeation chromatography, etc.) or weight average molecular weight (e.g., measured using light scattering, gel permeation chromatography, etc.) of the polymeric substance can be between about 10 kD and 10000 kD (e.g., 20 kD, 50 kD, 100 kD, 200 kD, 500 kD, 1000 kD, 2000 kD, 5000 kD, values or ranges therebetween, etc.). A polydispersity index (D) of the polymeric substance can have any value (e.g., 1-2, 1-3, 1-5, ≥1.5, ≥1, ≥2, etc.). However, the polymeric substance can have other suitable structural properties.

The polymeric substance preferably includes a polyvalent heteroatom (e.g., oxygen, sulfur, selenium, tellurium, nitrogen, phosphorous, arsenic, antimony, bismuth, boron, silicon, germanium, tin, etc.) in a backbone and/or off the backbone of the polymeric substance. Examples of polymeric substances with polyvalent heteroatoms in the backbone of the polymer include: polyester (e.g., polyethylene terephthalate, glycerine phthalate, poly(ethylene succinate), polybutylene adipate terephthalate, polybutylene succinate, polybutylene terephthalate (PBT), polycaprolactone, polycyclohexylenedimethylene terephthalate, polydiocanone, polyester resin, polyethylene furan-2,5-dicarboxylate, polytethylene naphthalate, polyglycolide or poly(glycolic acid), polyhydroxyalkanoates, polyhyroxybutyrate, polylactic acid, polytrimethylene terephthalate, poly(3-hydroxybutyrate-co-3-hydroxyvalerate), poly(lactic-co-glycolic acid), poly(bisphenol A terephthalate), poly(bisphenol A isophthalate), texteline, poly(methyl methacrylate) (PMMA), copolyester, Vectran, alkyds, etc.), polycarbonate (e.g., poly(trimethylene carbonate), polypropylene carbonate, poly(allyl diglycol carbonate), poly(bisphenol A carbonate), poly(bisphenol C carbonate), tritan, etc.), poly(acrylic acid) (PAA or derivatives thereof), poly(methacrylic acid) (PMAA or derivatives thereof), polyether (e.g., polyoxymethylene (POM), polyethylene oxide, polyethylene glycol, polypropylene glycol, polypropylene oxide, polytetramethylene glycol, polytetrahydrofuran, polyfurfurol, polyphenyl ether, poly(p-phenylene oxide), poly(Bisphenol A-co-epichlorohydrin), etc.), polyetherketone (e.g., polyaryletherketone, polyetheretherketones, polyetherketoneketone, polyetheretherketoneketone, polyetheketoneetherketoneketone, etc.), polyimide (e.g., Apical, Kapton, UPILEX, VTEC PI, Norton TH, Kaptrex, P84 NT, Meldin, Vespel, Plavis, etc.), polyamide (e.g., nylon 6, nylon 66, nylon 46, nylon 410, nylon 510, nylon 4T, nylon D6, nylon DT, nylon DI, nylon 610, nylon 612, nylon 6T, nylon 6I, nylon MXD6, mylon 9T, nylon 1010, nylon 10T, nylon1212, nylon 12T, nylon PACM12, nylon TMDT, nylon 11, nylon 12, nylon 1,6, nylon copolymers, nylon blends, etc.; aramid such as Kevlar, Technora, Twaron, Heracron, Nomex, Teijinconex, Innegra S, etc.; polyphthalamide (PPA); etc.), polyamide imide (PAI or derivatives thereof), polyetherimides (PEI or derivatives thereof), polyamine (e.g., polyethylenimine, polyaniline, polypyrrole, etc.), polysulfone (e.g., polyether sulfone (PES), poly(phenylene sulfone) (PPSU), poly(arylene sulfone), poly(bisphenol-A sulfone), Victrex HTA, etc.), polythiophenes (e.g., poly(3,4-ethylenedioxythiophene), polyselenides, polyureas (e.g., spandex, Line-X, GLS 100R, Pentens SPU-1000, etc.), polysiloxanes (e.g., polydimethylsiloxane, polymethylhydrosiloxane, polysilicone-15, PTC rubber, etc.), polysulfides (e.g., poly(p-phenylene-sulphide) (PPS), within vulcanized rubber, etc.), polyurethane (e.g., Sorbothane, poly(p-phenylene-2,6-benzobisoxazonle), polyurethanes derived from tolune-2,4-diisocyanate, polyurethanes derived from 4,4′-methylene diphenyl diisocyanate, polyurethanes derived from hexamethylene diisocyanate, polyurethanes derived from isophorone diisocyanate, etc.), semi-synthetic polymers (e.g., modified biopolymers, rayon, etc.), polybenzimidazole (PBI), melamine resins, polybezoxazines, polyhexahydrotriazine, polyisocyanurate, polymer blends or combinations (e.g., of two or more of the preceding polymers, copolymers of one or more of the preceding polymers with one or more of the following polymers, etc.), and/or other suitable polymers. Examples of polymers with heteroatoms off the backbone of or within a pendant group of the polymer include: polyvinyl alcohol (PVA); poly(4-vinylphenol); polyvinyl acetate (PVac); polyvinyl acetals such as polyvinyl butyral (PVB), polyvinyl formal (PVF), etc.; polyvinyl nitrate (PVN); poly(N-vinylacetamide); polyvinyl acetate phthalate (PVAP); polyvinylpyrrolidone; polyacrylonitrile; polyketones (such as ethylene-carbon monoxide co-polymer); polyacrylates; polymethacrylates; polycyanoacrylates; polyfluorene; polyallylamine, polystyrene sulfonate; polyvinylpolypyrrolidone (PVPP); polyacrylamide; acrylonitrile butadiene styrene (ABS); styrene acrylonitrile resin (SAN); phenol formaldehyde resins (such as Bakelite, novolak, polyoxybenzylmethyleneglycolanhyrdide, resoles, etc.); and/or other suitable polymers.

However, the polymeric substance can include a monovalent heteroatom (e.g., fluorine, chlorine, bromine, iodine, etc, particularly as substituents off the backbone of, within a pendant group of, etc. the polymer such as polychloroprene, poly(vinyl chloride), polytetafluoroethylene, polychlorotrifluoroethylene, polyvinyl fluoride, polyvinylidene fluoride, perfluoroalkoxy polymer, perfluorinated elastomer, perfluoropolyether, etc.), and/or hydrocarbon backbone and pendant groups (e.g., polyolefins such as polyethylene, polypropylene, polymethylpentene, polybutene-1, polybutylene, ethylene-octene copolymers, stereo-block polypropylene, olefine block copolymers, propylene-butane copolymers, polyisobutylene, poly(α-olefins), ethylene propylene rubber, ethylene propylene diene monomer rubber, polystyrene, styrene butadiene rubber, etc.; nitrile rubber; polyenes such as polyethyne, cuprene, nitrile rubber, polyisoprene, polybutadiene, etc.; poly(p-phenylene); poly(p-phenylene vinylene); etc.).

In some variants, copolymers (particularly but not exclusively between a polyester and one or more other polymers including other polyesters) can be preferred for the polymeric substance. These variants can have a technical advantage of leveraging a polymer degradant that works for one constituent polymer (or monomer) of the copolymer to degrade the remaining constituents (for instance by facilitating a favorable interaction geometry within an active site of the polymer degradant).

The polymeric substance preferably includes the composition (e.g., the polymer degradant and the nano-protectant) within the polymeric substance. A concentration of the composition (and/or polymer degradant or nano-protectant) within the polymeric substance can depend on a target degradation rate; degradation conditions; polymeric substance half-life; the polymer degradant; the nano-protectant; the polymeric substance; a use of the polymeric substance (e.g., a target lifetime based on the use case of the polymeric substance); physical, mechanical, chemical, and/or other such properties of the polymeric substance (e.g., include an amount of the composition such that the polymeric substance properties are substantially unchanged); and/or can otherwise depend on properties of the composition and/or polymeric substance. For instance, the concentration of the composition (e.g., combined weight, combined volume, individual weight, individual volume, combined particle count, individual particle count, etc. of polymer degradant and/or nano-protectant) can be between about 0.01% and 10% (e.g., wt %, vol %, stoichiometric percent, etc.) of the polymeric substance. However, the concentration can be less than 0.01% or greater than 10% (e.g., depending on the use cases).

The polymer degradant preferably functions to degrade the polymeric substance (and/or a plurality of polymeric substances). The polymer degradant preferably degrades the polymeric substance into monomers and/or other polymer precursors (e.g., where the monomers and/or polymer precursors can be reused such as to generate more of the polymeric substance and/or a new polymeric substance) such as by processively (e.g., monomer by monomer) breaking down the polymeric substance starting from an end of the polymeric substance. The processive degradation can provide a technical advantage of avoiding the generation of microplastics. However, the polymer degradant can additionally or alternatively degrade the polymeric substance into nonpolymeric precursors (e.g., other chemical species that can be used and/or leveraged for further processes), carbon, carbon monoxide, carbon dioxide, water, oligomers (e.g., with a maximum chain length, that can be reused for polymeric substance production, etc.), and/or into any suitable species.

The polymer degradant is preferably an enzyme (or combination of enzymes). However, additionally or alternatively, a chemical degradant (e.g., acid, base, oxidizing agent, reducing agent, etc.) and/or any suitable degradant can be used.

The polymer degradant is preferably chosen based on the polymeric substance (e.g., polymer class, polymer family, subunits of the polymer, monomers, backbone structure, polymerization mechanism, etc.) that is to be degraded. However, the polymer degradant can be a general use polymer degradant (e.g., can work for a plurality of polymeric substances), can depend on degradation conditions (e.g., predicted degradation conditions), and/or can otherwise be chosen.

Examples of enzymatic polymer degradants include: amidases (e.g., acrylamide amidohydrolase, acrylamidase, acylase, amidohydrolase, deaminase, fattyacylamidase, N-acetylaminohydrolase, peptide amidase, fatty acid amide hydrolase, malonamidase E2, Glu-tRNA (Gln) amidotransferase, 6-aminohexanoate-cyclic-dimer hydrolase, 6-aminohexanoate-dimer hydrolase, 6-aminohexanoate-oligomer endohydrolase, etc.), lyases (e.g., decarboxylases in the classification EC 4.1.1, aldehyde lyases in the classification EC 4.1.2, oxo acid lyases in the classification EC 4.1.3, lyases in the classification EC 4.1.99, lyases in the classification EC4.2 such as dehydratases or hydro-lyases, lyases in the classification EC4.3, lyases in the classification EC4.4, lyases in the classification EC4.5, lyases in the classification EC 4.6, lyases in the classification 4.99, etc.), hydrolases (e.g., with classification number EC 3.1 such as esterases, nucleases, phosphodiesterases, lipases, phosphatases, DNAse, RNAse, cellulases, etc.: EC 3.2 such as DNA glycosylases, glycoside hydrolases, cellulases, etc.; EC 3.3; EC 3.4 such as proteases, peptidases, trypsins, subtilisins, elastases, papains, etc.; EC 3.5 such as ureases; EC 3.6 such as acid anhydride hydrolases, helicases, GTPase, etc.; EC 3.7; EC 3.8; EC 3.9; EC 3.10; EC 3.11; EC 3.12; EC 3.13; etc.; hydrolases acting on ester bonds in the class EC 3.1, including lipases; hydrolases acting on carbon-nitrogen bonds other than peptide bonds EC 3.5 such as bonds in linear amides EC 3.5.1; etc.), ligases (e.g., with classification number EC 6.1; with classification number EC 6.2; with classification number EC 6.3 such as argininosuccinate synthetase; with classification number EC 6.4 such as acetyl-CoA carboxylase; with classification number EC 6.5 such as DNA ligase; with classification number EC 6.6 such as chelatases; etc.), transferases (e.g., with classification number EC 2.1 such as methyltransferase, formyltransferase, etc.; with classification number EC 2.2 such as transketolase, transaldolase, etc.; with classification number EC 2.3 such as acyltransferase; with classification number EC 2.4 such as glycosyltransferase, hexosyltransferase, pentosyltransferase, etc.; with classification number EC 2.5 such as riboflavin synthase, chlorophyll synthase, etc.; with classification number EC 2.6 such as transaminase, oximinotransferase, etc.; with classification number EC 2.7 such as phosphotransferase, polymerase, kinase, etc.; with classification number EC 2.8 sulfurtransferase, sulfotransferase, etc.; with classification number EC 2.9 such as selenotransferase; with classification number EC 2.10 such as molybdenumtransferase, tungstentransferase, etc.; etc.), isomerases (e.g., with classification number EC 5.1 such as racemases, epimerases, etc.; with classification number EC 5.2 such as cis-trans isomerases; with classification number EC 5.3 such as intramolecular oxidoreductases; with classification number EC 5.4 such as intramolecular transferases; with classification number EC 5.5 intramolecular lyases; etc.), oxidoreductases (e.g., with classification number EC 1.1 such as alcohol reductases, methanol dehydrogenase, etc.; with classification number EC1.2; with classification number EC 1.3 such as CH—CH oxidoreductases; with classification number EC 1.4 such as amino acid oxidoreductases, monoamine oxidase, etc.; with classification number EC 1.5; with classification number EC 1.6; with classification number EC 1.7; with classification number EC 1.8; with classification number EC 1.9; with classification number EC 1.10 such as laccases; with classification number EC 1.11 such as peroxidases; with classification number EC 1.12; with classification number EC 1.13 such as oxygenases, dioxygenases, latex clearing proteins, etc.; with classification number EC 1.14 such as alkane oxygenases, alkane monooxygenases, alkane hydroxylases, etc.; with classification number EC 1.15; with classification number EC 1.16; with classification number EC 1.17; with classification number EC 1.18; with classification number EC 1.19; with classification number EC 1.20; with classification number EC 1.21; oxidases; reductases; etc.), translocases (e.g., with classification EC 7.1 such as cytochromes; with classification EC 7.2; with classification EC 7.3; with classification EC 7.4; with classification EC 7.5; with classification EC 7.6; etc.), hydroxylases, peptidases (e.g., in the class EC 3.4; endopeptidases; exopeptidases; serin endopeptidases in the class EC 2.4.21 such as subtilisin, keratinase, proteinase K, etc.; etc.), PETase (e.g., PET hydrolase, tannase, MHETase, BHETase, etc.), esterases (e.g., LC-cutinase, TfCut2, Tcur1278, Tcuro390, etc.), carboxylesterases, polyurethanases, cutinases (e.g., enzymes in classification EC3.1.1.74), oxidative enzymes, enzymes that produce oxidative species (e.g., where the oxidative species can degrade the polymeric species), RNAse, DNAse, proteinase (e.g., Proteinase K), enzymes derived from microorganisms (e.g., associated with mealworms gut microbiome, fungi, etc.), lipases, and/or other suitable enzymes can be used. Note that while some of the listed enzymes may not directly degrade a polymeric substance, their inclusion can facilitate the degradation of the polymeric substance by an additional enzyme and hence can still be included as enzymatic polymeric degradants.

The nano-protectant functions to protect the polymer degradant (e.g., from processing conditions used in the preparation of the polymeric substance, from operating conditions of the polymeric substance, from transport conditions of the polymeric substance, etc.). However, the nano-protectant can additionally or alternatively function to solubilize the polymer degradant (e.g., in processing solution for the polymeric substance or precursors thereof, in the polymeric substance, etc.), protect the polymeric substance from the polymer degradant (e.g., act as a barrier between the polymer degradant and the polymeric substance until a target condition is achieved), protect the polymer degradant from itself (e.g., from other molecules of the polymer degradant), modify behavior of the polymer degradant (e.g., substrate specificity, substrate processivity, etc.), create a microenvironment (e.g., a favorable microenvironment for interaction of the polymeric substance and the polymer degradant), and/or can otherwise function.

The nano-protectant can fully encapsulate (e.g., surround) the polymer degradant, can partially encapsulate the polymer degradant (e.g., surround, enclose, etc. an active site of the polymer degradant), and/or can otherwise be arranged relative to the polymer degradant. As a first specific example, a nanoprotectant can be designed based on a total surface hydrophobicity and/or hydrophilicity of a polymer degradant. As a second specific example, a nanoprotectant can be designed based on a total surface charge of a polymer degradant. As a third specific example, a nanoprotectant can be designed based on a total active area hydrophobicity and/or hydrophilicity of an active site of a polymer degradant. As a fourth specific example, a nanoprotectant can be designed based on a total active area charge of a polymer degradant. However, a nano-protectant can otherwise be designed. The nano-protectant can interact with the polymer degradant chemically (e.g., forming one or more chemical bonds with the polymer degradant such as disulfide bonds, covalent bonds, etc.), physically (e.g., electrostatic interaction, hydrogen bonding, Van der Waals force, London dispersion forces, Debye forces, Keesom force, hydrophobic/hydrophilic effect, etc.), and/or can otherwise interact with (or not interact with) the polymer degradant. In some variants, the nano-protectant and the polymer degradant can spontaneously interact (e.g., the nano-protectant can encapsulate the polymer degradant spontaneously in solvent upon mixing). However, the interaction between the polymer degradant and the nano-protectant can be promoted (e.g., using heat, solvent transfer, sonication, addition of chemical species, etc.) and/or can otherwise occur.

The nano-protectant is preferably a polymeric protectant. However, the nano-protectant can additionally or alternatively include an inorganic protectant (e.g., a hollow, porous, solid, etc. particle; inorganic polymer; etc. that can contain, absorb, adsorb, etc. the polymer degradant, where the inorganic protectant can for instance be degraded to release the polymer degradant), peptoids and/or cyclic polymers (e.g., that confer multiple functionalities such as hydrophobicity and hydrophilicity), and/or using any suitable protectant.

The polymeric protectant can be a statistical heteropolymer (e.g., a polymer formed from a combination of 2 or more different monomers with the monomers distributed according to a statistical distribution such as based on the reaction kinetics of the monomers, markovian statistics, etc.; random polymer; etc.). However, the polymeric protectant can additionally or alternatively include a block copolymer, patterned copolymers (e.g., alternating polymer; polymer with an engineered, intentional, etc. arrangement of monomers; gradient copolymers; crosslinked polymer; etc.), periodic copolymers, stereoblock copolymers, branched copolymer (e.g., graft copolymer, brush copolymer, comb copolymer, ladder polymer, star copolymer, etc.), homopolymer (e.g., made from a zwitterionic, amphiphilic, etc. monomer), and/or can have any suitable structure.

The polymeric protectant can include (e.g., be made from, be composed of, be composed essentially of, consist of, consist essentially of, etc.) one or more hydrophilic monomers, one or more hydrophobic monomers, one or more charged monomers (e.g., cations and/or anions at a target pH in a given solvent for a given temperature such as at pH 7 in water at about 25° C.), one or more zwitterionic monomers (e.g., monomers that include a cationic and anionic moiety at a target pH in a given solvent for a given temperature such as at pH 7 in water at about 25° C.), one or more neutral hydrophilic monomers (e.g., polar monomers), one or more aliphatic monomers, one or more aromatic monomers, one or more mechanically hindered monomers (e.g., a monomer having a sterically hindering group such as a t-butyl; a monomer containing a spiro, bicyclic, or other such structural motif; a monomer having ortho, meta, or other similar arrangements of polymerizable moieties such as not on opposing sides of the monomer; etc.), and/or can include any suitable monomer(s). In variants that include a plurality of the same type of monomer (e.g., two or more hydrophobic monomers), the different monomers preferably have different HLB values). For instance, a polymeric protectant preferably includes a weakly hydrophobic monomer (e.g., a monomer with an HLB value between 7.5 and 10) and a highly hydrophobic monomer (e.g., a monomer with an HLB value less than 7.5).

The selection of and/or amount of (e.g., ratio, relative amount, absolute amount, target amount, etc.) of each monomer can be selected based on a hydrophilic-lipophilic balance of the target polymeric protectant (e.g., HLB value such as to achieve a target HLB value of between 0 and 20), based on a hydrophobicity and/or hydrophilicity of the resulting polymeric protectant (e.g., using a modified hydrophilicity or hydrophobicity scale, based on a 3D structure of the polymeric protectant, physiochemical properties, etc.), based on a predicted charge (at a target pH and temperature) of the polymeric protectant, based on the polymeric degradant (e.g., structure, physiochemical properties, HLB of the polymeric degradant, charge, charge of a region of the polymeric degradant, predicted or known structure based on amino acid residues, etc.), based on the polymeric substance to be degraded (e.g., an HLB value thereof, a structure thereof, etc.), and/or can otherwise be chosen.

The monomers are preferably acrylates and/or methacrylates. However, other suitable polymerization chemistries can be used such as cyanoacrylate, epoxy, imide, polyester (e.g., formed via condensation reactions such as between a carboxylic acid and diol, alcoholysis, acidolysis, alcoholysis of acyl halide, etc.), polyether (e.g., formed by ring-opening polymerization of cyclic ethers, polycondensation of diols, etc.), polyamide (e.g., via a condensation reaction between a diamine and acyl halide, between a diamine and carboxylic acid, via condensation reactions between the amine and the carboxylate of molecules containing both moieties, via ring opening polymerizations of N-carboxyanhydrides, etc.), vinyl ether (e.g., to form polyvinyl ethers), vinyl esters (e.g., to form polyvinyl esters), aminocarboxylic acids, aminopolycarboxylic acids, hydroxycarboxylic acids, and/or thiol (e.g., for thiol-ene reaction) functional groups; where variants that use chemistries other than acrylate or methacrylate can be beneficial in variants where the polymeric substance to be degraded is an acrylate or methacrylate derivative, or where it may be desirable or critical for the degradant to degrade the protectant along with the polymeric substance once the degradation conditions are met. Typically, a polymeric nanoprotectant will not mix acrylates and methacrylates (because of differences in the average reaction rates for the different monomers). However, in some variants (e.g., by controlling concentrations, monomer introduction time, etc.) the polymeric protectant can include a combination of acrylates and methacrylates.

Examples of monomers that can be used to form a polymeric protectant include: acrylates (e.g., as shown for example in FIG. 2A where R can include linear or branching alkanes with between 0 and 20 carbons, cyclic or polycyclic alkanes with between 3 and 20 carbons, aromatic moieties, oligomers, optionally including heteroatoms such as nitrogen, oxygen, sulfur, selenium, boron, phosphorous, silicon, etc.; such as acrylic acid; methyl acrylate; ethyl acrylate; n-propyl acrylate; i-propyl acrylate; n-butyl acrylate; i-butyl acrylate; t-butyl acrylate; pentyl acrylate; hexyl acrylate; heptyl acrylate; octyl acrylate; nonyl acrylate; decyl acrylate; benzyl acrylate; methylbenzyl acrylate; tetrahydrobenzyl acrylate; hexahydrobenzyl acrylate; 2-ethylhexyl acrylate; acrylate polyethylene glycol (PEG) oligomer such as with a PEG molecular weight between about 100 Da and 2000 Da; acrylate polypropylene glycol (PPG) oligomer such as with a PPG molecular weight between about 100 Da and 2000 Da; acrylate polybutylene glycol (PBG) oligomer such as with a PBG molecular weight between about 100 Da and 2000 Da; acrylate PEG carboxylic acid; sodium acrylate; potassium acrylate; sulfopropyl acrylate salt; sulfoethyl acrylate salt; sulfomethyl acrylate salt; sulfo (2-methylpropyl) acrylate salt; acrylo t-butyl sulfonic acid; acrylate carboxylates such as including an alkali metal, ammonium, alkyl ammonium, etc. counterion; acrylate sulfates such as including an alkali metal, ammonium, alkyl ammonium, etc. counterion; acrylate phosphates such as including an alkali metal, ammonium, alkyl ammonium, etc. counterion; acrylate amines such as including an halide, sulfate, etc. counterion; 2-aminoethyl acrylate; 3-aminopropyl acrylate; N-[3-(dimethylamino) propyl]-2-acrylate; 3-acrylopropyl trimethylammonium salt; acryloyl oxyethyl dimethylbenzyl ammonium salt; acryloyl oxyethyl dimethyl ethyl ammonium salt; acryloyl oxyethyl trimethyl ammonium salt; acryloyl oxyethyl triethyl ammonium; amine modified acrylates; urethane acrylate oligomers; epoxy acrylate oligomers; polyester acrylate oligomers; polycarboxylate acrylate oligomers; etc.), cyanoacrylates (e.g., cyanoacrylic acid; methyl cyanoacrylate; ethyl cyanoacrylate; n-propyl cyanoacrylate; i-propyl cyanoacrylate; n-butyl cyanoacrylate; i-butyl cyanoacrylate; t-butyl cyanoacrylate; pentyl cyanoacrylate; hexyl cyanoacrylate; heptyl cyanoacrylate; octyl cyanoacrylate; nonyl cyanoacrylate; decyl cyanoacrylate; benzyl cyanoacrylate; methylbenzyl cyanoacrylate; tetrahydrobenzyl cyanoacrylate; hexahydrobenzyl cyanoacrylate; 2-ethylhexyl cyanoacrylate; cyanoacrylate polyethylene glycol (PEG) oligomer such as with a PEG molecular weight between about 100 Da and 2000 Da; cyanoacrylate polypropylene glycol (PPG) oligomer such as with a PPG molecular weight between about 100 Da and 2000 Da; cyanoacrylate polybutylene glycol (PBG) oligomer such as with a PBG molecular weight between about 100 Da and 2000 Da; cyanoacrylate PEG carboxylic acid; sodium cyanoacrylate; potassium cyanoacrylate; sulfopropyl cyanoacrylate salt; sulfoethyl cyanoacrylate salt; sulfomethyl cyanoacrylate salt; sulfo (2-methylpropyl) cyanoacrylate salt; cyanoacrylo t-butyl sulfonic acid; cyanoacrylate carboxylates such as including an alkali metal, ammonium, alkyl ammonium, etc. counterion; cyanoacrylate sulfates such as including an alkali metal, ammonium, alkyl ammonium, etc. counterion; cyanoacrylate phosphates such as including an alkali metal, ammonium, alkyl ammonium, etc. counterion; cyanoacrylate amines such as including an halide, sulfate, etc. counterion; 2-aminoethyl cyanoacrylate; 3-aminopropyl cyanoacrylate; N-[3-(dimethylamino) propyl]-2-cyanoacrylate; 3-cyanoacrylopropyl trimethylammonium salt; cyanoacryloyl oxyethyl dimethylbenzyl ammonium salt; cyanoacryloyl oxyethyl dimethyl ethyl ammonium salt; cyanoacryloyl oxyethyl trimethyl ammonium salt; cyanoacryloyl oxyethyl triethyl ammonium; amine modified cyanoacrylates; urethane cyanoacrylate oligomers; epoxy cyanoacrylate oligomers; polyester cyanoacrylate oligomers; etc.), methacrylates (e.g., as shown for example in FIG. 2B where R can include linear or branching alkanes with between 0 and 20 carbons, cyclic alkanes with between 3 and 20 carbons, aromatic moieties, oligomers, optionally including heteroatoms such as nitrogen, oxygen, sulfur, selenium, etc.; such as methacrylic acid; methyl methacrylate; ethyl methacrylate; n-propyl methacrylate; i-propyl methacrylate; n-butyl methacrylate; i-butyl methacrylate; t-butyl methacrylate; pentyl methacrylate; hexyl methacrylate; heptyl methacrylate; octyl methacrylate; nonyl methacrylate; decyl methacrylate; benzyl methacrylate; methylbenzyl methacrylate; tetrahydrobenzyl methacrylate; hexahydrobenzyl methacrylate; 2-ethylhexyl methacrylate; methacrylate polyethylene glycol (PEG) oligomer such as with a PEG molecular weight between about 100 Da and 2000 Da; methacrylate polypropylene glycol (PPG) oligomer such as with a PPG molecular weight between about 100 Da and 2000 Da; methacrylate polybutylene glycol (PBG) oligomer such as with a PBG molecular weight between about 100 Da and 2000 Da; methacrylate PEG carboxylic acid; sodium methacrylate; potassium methacrylate; sulfopropyl methacrylate salt; sulfoethyl methacrylate salt; sulfomethyl methacrylate salt; sulfo (2-methylpropyl) methacrylate salt; methacrylate t-butyl sulfonic acid; methacrylate carboxylates such as including an alkali metal, ammonium, alkyl ammonium, etc. counterion; methacrylate sulfates such as including an alkali metal, ammonium, alkyl ammonium, etc. counterion; methacrylate phosphates such as including an alkali metal, ammonium, alkyl ammonium, etc. counterion; methacrylate amines such as including an halide, sulfate, etc. counterion; 2-aminoethyl methacrylate; 3-aminopropyl methacrylate; N-[3-(dimethylamino) propyl]-2-methacrylate; 3-methacrylopropyl trimethylammonium salt; methacryloyl oxyethyl dimethylbenzyl ammonium salt; methacryloyl oxyethyl dimethyl ethyl ammonium salt; methacryloyl oxyethyl trimethyl ammonium salt; methacryloyl oxyethyl triethyl ammonium; amine modified methacrylates; urethane methacrylate oligomers; epoxy methacrylate oligomers; polyester methacrylate oligomers; etc.), acrylamides (e.g., as shown for example in FIG. 2C where R can include linear or branching alkanes with between 0 and 20 carbons, cyclic alkanes with between 3 and 20 carbons, aromatic moieties, oligomers, optionally including heteroatoms such as nitrogen, oxygen, sulfur, selenium, etc.; such as methyl acrylamide; ethyl acrylamide; n-propyl acrylamide; i-propyl acrylamide; n-butyl acrylamide; i-butyl acrylamide; t-butyl acrylamide; pentyl acrylamide; hexyl acrylamide; heptyl acrylamide; octyl acrylamide; nonyl acrylamide; decyl acrylamide; benzyl acrylamide; methylbenzyl acrylamide; tetrahydrobenzyl acrylamide; hexahydrobenzyl acrylamide; 2-ethylhexyl acrylamide; acrylamide polyethylene glycol (PEG) oligomer such as with a PEG molecular weight between about 100 Da and 2000 Da; acrylamide polypropylene glycol (PPG) oligomer such as with a PPG molecular weight between about 100 Da and 2000 Da; acrylamide polybutylene glycol (PBG) oligomer such as with a PBG molecular weight between about 100 Da and 2000 Da; acrylamide PEG carboxylic acid; sulfopropyl acrylamide salt; sulfoethyl acrylamide salt; sulfomethyl acrylamide salt; sulfo (2-methylpropyl) acrylamide salt; acrylamide t-butyl sulfonic acid; acrylamide carboxylates such as including an alkali metal, ammonium, alkyl ammonium, etc. counterion; acrylamide sulfates such as including an alkali metal, ammonium, alkyl ammonium, etc. counterion; acrylamide phosphates such as including an alkali metal, ammonium, alkyl ammonium, etc. counterion; acrylamide amines such as including an halide, sulfate, etc. counterion; 2-aminoethyl acryamide; 3-aminopropyl acrylamide; N-[3-(dimethylamino) propyl]-2-acrylamide; 3-acrylamide-propyl trimethylammonium salt; acrylamido oxyethyl dimethylbenzyl ammonium salt; acrylamido oxyethyl dimethyl ethyl ammonium salt; acrylamido oxyethyl trimethyl ammonium salt; acrylamido oxyethyl triethyl ammonium; amine modified acrylamides; urethane acrylamide oligomers; epoxy acrylamide oligomers; polyester acrylamide oligomers; etc.), methacrylamides (e.g., as shown for example in FIG. 2D where R can include linear or branching alkanes with between 0 and 20 carbons, cyclic alkanes with between 3 and 20 carbons, aromatic moieties, oligomers, optionally including heteroatoms such as nitrogen, oxygen, sulfur, selenium, etc.; such as methyl methacrylamide; ethyl methacrylamide; n-propyl methacrylamide; i-propyl methacrylamide; n-butyl methacrylamide; i-butyl methacrylamide; t-butyl methacrylamide; pentyl methacrylamide; hexyl methacrylamide; heptyl methacrylamide; octyl methacrylamide; nonyl methacrylamide; decyl methacrylamide; benzyl methacrylamide; methylbenzyl methacrylamide; tetrahydrobenzyl methacrylamide; hexahydrobenzyl methacrylamide; 2-ethylhexyl methacrylamide; methacrylamide polyethylene glycol (PEG) oligomer such as with a PEG molecular weight between about 100 Da and 2000 Da; methacrylamide polypropylene glycol (PPG) oligomer such as with a PPG molecular weight between about 100 Da and 2000 Da; methacrylamide polybutylene glycol (PBG) oligomer such as with a PBG molecular weight between about 100 Da and 2000 Da; methacrylamide PEG carboxylic acid; sulfopropyl methacrylamide salt; sulfoethyl methacrylamide salt; sulfomethyl acrylamide salt; sulfo (2-methylpropyl) methacrylamide salt; methacrylamide t-butyl sulfonic acid; methacrylamide carboxylates such as including an alkali metal, ammonium, alkyl ammonium, etc. counterion; methacrylamide sulfates such as including an alkali metal, ammonium, alkyl ammonium, etc. counterion; methacrylamide phosphates such as including an alkali metal, ammonium, alkyl ammonium, etc. counterion; methacrylamide amines such as including an halide, sulfate, etc. counterion; 2-aminoethyl methacrylamide; 3-aminopropyl methacrylamide; N-[3-(dimethylamino) propyl]-2-methacrylamide; 3-methacrylamide-propyl trimethylammonium salt; methacrylamido oxyethyl dimethylbenzyl ammonium salt; methacrylamido oxyethyl dimethyl ethyl ammonium salt; methacrylamido oxyethyl trimethyl ammonium salt; methacrylamido oxyethyl triethyl ammonium; amine modified methacrylamides; urethane methacrylamide oligomers; epoxy methacrylamide oligomers; polyester acrylamide oligomers; etc.), esters, amides, olefins, carboxyanhydrides, epoxides, amino acids (e.g., canonical amino acids, nonproteinogenic amino acids, unnatural amino acids, etc.), and/or other suitable monomers.

As a first illustrative example, a polymeric protectant can include a stochastic mixture of a hydrophobic monomer (e.g., methyl acrylate, ethyl acrylate, n-propyl acrylate, i-propyl acrylate, n-butyl acrylate, i-butyl acrylate, t-butyl acrylate, pentyl acrylate, hexyl acrylate, heptyl acrylate, octyl acrylate, nonyl acrylate, decyl acrylate, lauryl acrylate, stearyl acrylate, 2-ethylhexyl acrylate, isobornyl acrylate, methyl methacrylate, ethyl methacrylate, n-propyl methacrylate, i-propyl methacrylate, n-butyl methacrylate, i-butyl methacrylate, t-butyl methacrylate, pentyl methacrylate, hexyl methacrylate, heptyl methacrylate, octyl methacrylate, nonyl methacrylate, decyl methacrylate, 2-ethylhexyl methacrylate, isobornyl methacrylate, etc.), a cationic monomer (e.g., 2-amino-ethyl acrylate, 3-aminopropyl acrylate, 2-(dimethylamino)ethyl acrylate, 2-(diethylamino)ethyl acrylate, N-[3-(dimethylamino) propyl]-2-acrylate, 3-acrylopropyl trimethylammonium salt, acryloyl oxyethyl dimethylbenzyl ammonium salt, acryloyl oxyethyl dimethyl ethyl ammonium salt, acryloyl oxyethyl trimethyl ammonium salt, acryloyl oxyethyl triethyl ammonium, 2-aminoethyl methacrylate, 3-aminopropyl methacrylate, 2-(dimethylamino)ethyl methacrylate, 2-(diethylamino)ethyl methacrylate N-[3-(dimethylamino) propyl]-2-methacrylate, 3-methacrylopropyl trimethylammonium salt, methacryloyl oxyethyl dimethylbenzyl ammonium salt, methacryloyl oxyethyl dimethyl ethyl ammonium salt, methacryloyl oxyethyl trimethyl ammonium salt, methacryloyl oxyethyl triethyl ammonium, etc.), and a polar monomer (e.g., ethoxy diethylene glycol acrylate, methoxy triethylene glycol acrylate, methoxy dipropylene glycol acrylate, phenoxyethyl acrylate, phenoxydiethylene glycol acrylate, phenoxy polyethylene glycol acrylate, m-phenoxybenzyl acrylate, 2-hydroxyl butyl acrylate, 2-hydroxy-3-phenoxypropyl acrylate, 2-acryloloxyethyl succinate, 2-acryloyloxyethyl hexahydro phthalate, 2-acryloyloxyethyl phthalate, 2-acryloyloxyethyl 2-hydroxyethyl phthalate, triethylene glycol diacrylate, tetrahydrobenzyl acrylate, hexahydrobenzyl acrylate, hydroxyethyl acrylate, acrylate polyethylene glycol (PEG) oligomer such as with a PEG molecular weight between about 100 Da and 2000 Da, acrylate polypropylene glycol (PPG) oligomer such as with a PPG molecular weight between about 100 Da and 2000 Da, acrylate polybutylene glycol (PBG) oligomer such as with a PBG molecular weight between about 100 Da and 2000 Da, ethoxy diethylene glycol methacrylate, methoxy triethylene glycol methacrylate, methoxy dipropylene glycol methacrylate, phenoxyethyl methacrylate, phenoxydiethylene glycol methacrylate, phenoxy polyethylene glycol methacrylate, m-phenoxybenzyl methacrylate, 2-hydroxyl butyl methacrylate, 2-hydroxy-3-phenoxypropyl methacrylate, 2-methacryloloxyethyl succinate, 2-methacryloyloxyethyl phthalate, 2-methacryloyloxyethyl phthalate, 2-methacryloyloxyethyl 2-hydroxyethyl phthalate, triethylene glycol tetrahydrobenzyl methacrylate, hexahydrobenzyl methacrylate, hydroxyethyl methacrylate, methacrylate polyethylene glycol (PEG) oligomer such as with a PEG molecular weight between about 100 Da and 2000 Da, methacrylate polypropylene glycol (PPG) oligomer such as with a PPG molecular weight between about 100 Da and 2000 Da, methacrylate polybutylene glycol (PBG) oligomer such as with a PBG molecular weight between about 100 Da and 2000 Da, etc.). As a second illustrative example, a polymeric protectant can include a stochastic mixture of a hydrophobic monomer (e.g., as described above or below), an anionic monomer (e.g., sodium acrylate; potassium acrylate; sulfopropyl acrylate salt; sulfoethyl acrylate salt; sulfomethyl acrylate salt; sulfo (2-methylpropyl) acrylate salt; acrylo t-butyl sulfonic acid; carboxyethyl acrylate; mono-2-(acryloxy)ethyl succinate; acrylate carboxylates such as including an alkali metal, ammonium, alkyl ammonium, etc. counterion; acrylate sulfates such as including an alkali metal, ammonium, alkyl ammonium, etc. counterion; acrylate phosphates such as including an alkali metal, ammonium, alkyl ammonium, etc. counterion; acrylate nitrates such as including an alkali metal, ammonium, alkyl ammonium, etc. counterion; sodium methacrylate; potassium methacrylate; sulfopropyl methacrylate salt; sulfoethyl methacrylate salt; sulfomethyl methacrylate salt; sulfo (2-methylpropyl) methacrylate salt; methacrylo t-butyl sulfonic acid; carboxyethyl methacrylate; mono-2-(methacryloxy)ethyl succinate; methacrylate carboxylates such as including an alkali metal, ammonium, alkyl ammonium, etc. counterion; methacrylate sulfates such as including an alkali metal, ammonium, alkyl ammonium, etc. counterion; methacrylate phosphates such as including an alkali metal, ammonium, alkyl ammonium, etc. counterion; methacrylate nitrates such as including an alkali metal, ammonium, alkyl ammonium, etc. counterion; etc.), and a polar monomer (e.g., as described above or below). As a third illustrative example, a polymeric protectant can include a stochastic mixture of a hydrophobic monomer (e.g., as described above or below), a cationic monomer (e.g., as described above or below), and an anionic monomer (e.g., as described above or below). As a fourth illustrative example, a polymeric protectant can include a stochastic mixture of a hydrophobic monomer (e.g., as described above or below), a charged monomer (e.g., an anionic monomer, a cationic monomer, a mixture of the two for example as described above or below), and an aromatic monomer (e.g., phenyl acrylate, benzyl acrylate, methylbenzyl acrylate, 2-napthyl acrylate, 3-napthyl acrylate, 3-(4-methoxy-1-nahthyl) acrylate, 9-anthracenylmethyl acrylate, 1-pyrenemethyl acrylate, phenoxyethyl acrylate, phenoxydiethylene glycol acrylate, phenoxy polyethylene glycol acrylate, m-phenoxybenzyl acrylate, phenyl methacrylate, benzyl methacrylate, methylbenzyl methacrylate, 2-napthyl methacrylate, 3-napthyl methacrylate, 3-(4-methoxy-1-nahthyl) methacrylate, 9-anthracenylmethyl methacrylate, 1-pyrenemethyl methacrylate, phenoxyethyl methacrylate, phenoxydiethylene glycol methacrylate, phenoxy polyethylene glycol methacrylate, m-phenoxybenzyl methacrylate, styrene, α-methylstyrene, etc.). As a fifth illustrative example, a polymeric protectant can include a stochastic mixture of a first hydrophobic monomer (e.g., with an hlb value greater than about 7.5 and less than about 10, with hydrocarbon chains that contain at most 3 carbon atoms such as methyl acrylate, ethyl acrylate, n-propyl acrylate, i-propyl acrylate, methyl methacrylate, ethyl methacrylate, n-propyl methacrylate, i-propyl methacrylate, etc.), a second hydrophobic monomer (e.g., with an hlb value less than about 7.5, with hydrocarbon side chains that contain 4 or more carbon atoms such as n-butyl acrylate, i-butyl acrylate, t-butyl acrylate, pentyl acrylate, hexyl acrylate, heptyl acrylate, octyl acrylate, nonyl acrylate, decyl acrylate, 2-ethylhexyl acrylate, lauryl acrylate, stearyl acrylate, isobornyl acrylate, n-butyl methacrylate, i-butyl methacrylate, t-butyl methacrylate, pentyl methacrylate, hexyl methacrylate, heptyl methacrylate, octyl methacrylate, nonyl methacrylate, decyl methacrylate, 2-ethylhexyl methacrylate, lauryl methacrylate, stearyl methacrylate, isobornyl methacrylate, etc.), and a polar monomer (e.g., as described above or below). As a sixth illustrative example, a polymeric protectant can include a stochastic mixture of a first hydrophobic monomer (e.g., with an hlb value greater than about 7.5 and less than about 10, with hydrocarbon chains that contain at most 3 carbon atoms, as described above or below, etc.), a second hydrophobic monomer (e.g., with an hlb value less than about 7.5, with hydrocarbon side chains that contain 4 or more carbon atoms, as described above or below, etc.), and a charged monomer (e.g., an anionic monomer, a cationic monomer, a combination of anionic and cationic monomers, as described above or below). As a seventh illustrative example, a polymeric protectant can include a stochastic mixture of a first hydrophobic monomer (e.g., with an hlb value greater than about 7.5 and less than about 10, with hydrocarbon chains that contain at most 3 carbon atoms, as described above or below, etc.), a second hydrophobic monomer (e.g., with an hlb value less than 7.5, with hydrocarbon side chains that contain 4 or more carbon atoms, as described above or below, etc.), and an aromatic monomer (as described above or below). As an eighth illustrative example, a polymeric protectant can include a stochastic mixture of a first hydrophobic monomer (e.g., with an hlb value greater than about 7.5 and less than about 10, with hydrocarbon chains that contain at most 3 carbon atoms, as described above or below, etc.), a second hydrophobic monomer (e.g., with an hlb value less than about 7.5, with hydrocarbon side chains that contain 4 or more carbon atoms, as described above or below, etc.), a polar monomer (e.g., as described above or below), and optionally a charged monomer (e.g., an anionic monomer, a cationic monomer, a mixture of the two, as described above or below, etc.). As a ninth illustrative example, a polymeric protectant can include a stochastic mixture of an aromatic monomer (e.g., as described above or below), a charged monomer (e.g., a cationic monomer, an anionic monomer, a combination of anionic and cationic monomers, as described above or below, etc.), and a polar monomer (e.g., as described above or below). As a tenth illustrative example, a polymeric protectant can include a stochastic mixture of an aromatic monomer (e.g., as described above or below), and a charged monomer (e.g., a cationic monomer, an anionic monomer, a combination of anionic and cationic monomers, as described above or below, etc.). As an eleventh illustrative example, a polymeric protectant can include a stochastic mixture of an aromatic monomer (e.g., as described above or below), and a polar monomer (e.g., as described above or below). As a twelfth illustrative example, a polymeric protectant can include a stochastic mixture of a hydrophobic monomer (e.g., as described above or below), and a charged monomer (e.g., a cationic monomer, an anionic monomer, a combination of anionic and cationic monomers, as described above or below, etc.). As a thirteenth illustrative example, a polymeric protectant can include a stochastic mixture of a hydrophobic monomer (e.g., as described above or below), and a polar monomer (e.g., as described above or below). As a fourteenth illustrative example, a polymeric protectant can include a stochastic mixture of a polar monomer (e.g., as described above or below), and a charged monomer (e.g., a cationic monomer, an anionic monomer, a combination of anionic and cationic monomers, as described above or below, etc.). As a fifteenth illustrative example, a polymeric protectant can include a stochastic mixture of a hydrophobic monomer (e.g., as described above or below), an aromatic monomer (e.g., as described above or below), a polar monomer (e.g., as described above or below), and a charged monomer (e.g., a cationic monomer, an anionic monomer, a combination of anionic and cationic monomers, as described above or below, etc.). In variations of the preceding fifteen illustrative examples, non-stochastic (e.g., engineered polymers such as block co-polymers, graft polymers, cross-linked polymers, alternating polymers, or other patterns of monomeric subunits within the polymeric protectant) can be used (e.g., with the same monomer composition as described). In related variations (that can be combined with the preceding variations), additional or alternative classes of monomers can be used (typically replacing all or part of the hydrophilic monomers such as polar monomers or charged monomers) such as silicon-containing monomers (e.g., silicates to facilitate interactions with silicones), thiol-containing monomers (e.g., to form disulphide bonds or interactions with polymer degradant), mechanically rigid monomers (e.g., to introduce structural variation or rigidness within a nanoprotectant), and/or other classes of monomer can be included. However, other suitable combinations of monomers can be used to form the polymeric protectant.

Typically, hydrophobic monomers (inclusive of weakly hydrophobic monomers, strongly hydrophobic monomers, aromatic monomers, or other monomers with an hlb value less than about 10) are a majority (e.g., >50% by stoichiometry up to and inclusive of 100% of the stoichiometry) of the polymer. The remainder of the polymer composition can be hydrophilic monomers (e.g., charged monomers, anionic monomers, cationic monomers, polar monomers, etc. such as monomers with an hlb value greater than about 10). However, as discussed above, the exact composition can be tailored based on the polymeric substance to be degraded and/or the polymer degradant and therefore the exact composition can vary (e.g., some variants can have a majority of, up to and including consisting of, hydrophilic monomer composition).

In a specific example, a polymeric nano-protectant can be made by polymerizing a mixture of about 20-80% (by moles) of a first weakly hydrophobic monomer (e.g., with an hlb between 7.5 and 10 such as methyl methacrylate (MMA)) which can function to control a hydrophobicity of the polymeric nano-protectant and/or confer flexibility or decrease an entropic penalty of the polymeric nano-protectant, about 5-50% (by moles) of a second, strongly hydrophobic monomer (e.g., with an hlb less than about 7.5 such as 2-ethylhexyl methacrylate (EHMA), n-butyl methacrylate (nBuMA), etc.) which can function to control a hydrophobicity and/or hydrophobic interactions of the polymeric nano-protectant and the polymer degradant, about 2-40% (by moles) of a polar monomer (e.g., oligo (ethylene glycol) methacrylate (OEGMA) such as with a molecular weight between about 100 Da and 2000 Da) which can function to confer hydrophilicity to the polymeric nano-protectant and/or can stabilize an (enzymatic) polymer degradant, about 0-10% (by moles) of a charged monomer (such as sulfopropyl methacrylate (SPMA)) which can function to mediate a charged interaction with a polymer degradant, where the percentages add up to 100%. In variations of this specific example, associated acyrlates (e.g., methyl acrylate (MA) instead of methyl methacrylate), acrylamides (e.g., methyl acrylamide instead of methyl methacrylate), methacrylamides (e.g., methyl methacrylamide instead of methyl methacrylate), combinations thereof, and/or other suitable monomers (e.g., n-carboxyanhydrides (NCAs)) can substitute all or a portion of the corresponding monomer(s) in the specific example (e.g., an about 20% MMA example could instead have about 10% MMA and about 10% MA, about 20% MA, etc.). In another variation of this specific example, anionic monomers can be replaced with (e.g., partially or fully) a cationic monomer (e.g., 2-aminoethyl methacrylate (AEMA) such as about 10% SPMA could additionally or alternatively be about 5% SPMA and about 5% AEMA, about 10% AEMA, etc.).

The polymeric protectant and polymer degradant preferably form clusters where the polymeric protectant surrounds, encapsulates, encloses, and/or otherwise forms a barrier around the polymer degradant. The clusters preferably have a size (e.g., average size, characteristic size, diameter, longest one-dimensional extent, etc.) between about 10 nm and 500 nm. However, the clusters can have other suitable sizes. Each cluster can include a plurality of polymer degradants. When a cluster includes a plurality of polymer degradants, the number of polymer degradants within each cluster is preferably less than a threshold number above which the polymer degradants begin to react with (and therefore degrade) each other. For instance, in one example, a cluster can include at most 8 polymer degradants within a cluster (e.g., when the polymer degradants form an approximate cubic appearing packing). However, the threshold number can otherwise be determined.

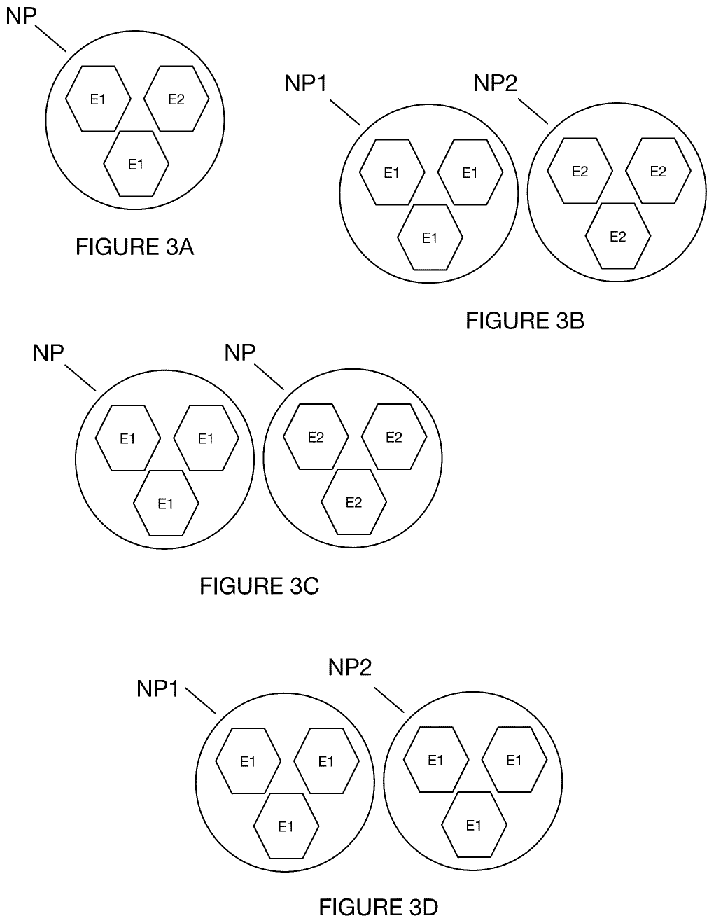

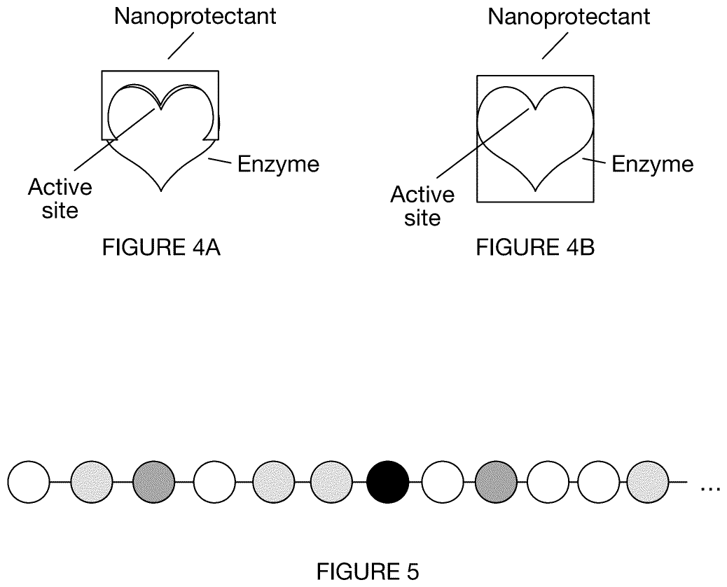

In formulations or compositions that include a plurality of polymer degradants, the plurality of polymer degradants can be included within the same cluster (as shown for example in FIG. 3A where NP refers to a nanoprotectant and E1 and E2 refer to a first and second polymer degradant), the plurality of polymer degradants can be separated into distinct clusters (as shown for example in FIG. 3C where NP refers to a nanoprotectant, and E1 and E2 refer to a first and second polymer degradant), a first plurality of polymer degradants can be included within cluster with a first nanoprotectant (e.g., first plurality of nanoprotectants) and a second plurality of polymer degradants (that can be the same as or different from the first plurality of polymer degradants) can be included within a second cluster with a second nanoprotectant (e.g., second plurality of nanoprotectants where the second plurality of nanoprotectants can be the same as or different from the first nanoprotectants or first plurality of nanoprotectants), and/or the polymer degradants can otherwise be arranged. In some variations, as shown for example in FIG. 3B, each polymer degradant can be included in a cluster formed with a distinct nano-protectant.

In some variations of the composition (as shown for example in FIG. 3D), the composition can include a plurality of clusters formed from different nano-protectants and/or a cluster can have a plurality of distinct nano-protectants. However a composition can include a single nano-protectant.

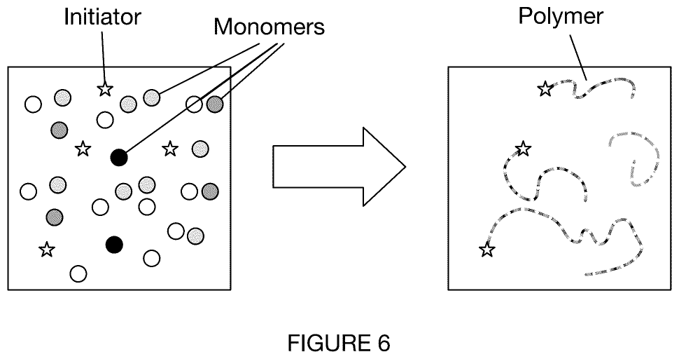

The polymeric protectant can be made using free radical polymerization (e.g., following an initiation, propagation, termination process; bulk polymerization, solution polymerization, suspension polymerization, emulsion polymerization, template polymerization, plasma polymerization, sonication, etc.), anionic polymerization, cationic polymerization, controlled radical polymerization (e.g., living free radical polymerization, reversible-deactivation radical polymerization, inferter polymerization such as using dithiocarbamate, stable free radical mediated polymerization, nitroxide mediated polymerization (NMP), verdazyl mediated polymerization (VMP), atom transfer radical polymerization (ATRP), reversible addition fragmentation chain transfer (RAFT) polymerization, iodine-transfer polymerization (ITP), selenium-centered radical-mediated polymerization, telluride-mediated polymerization (TERP), stibine-mediated polymerization, transition metal mediated polymerization, copper mediated polymerization, cobalt mediated radical polymerization, etc.), ring opening polymerizations, condensation polymerization and/or other suitable polymerization mechanism to polymerize the monomers. Variants that leverage free radical polymerization methods can be particularly beneficial as they can facilitate large quantities of polymeric nanoprotectant to be formed. However, as compared to living polymerization processes, free radical polymerization can result in greater heterogeneity (e.g., size dispersity, variance between individual polymers, as shown for example in FIG. 6, etc.) in the as formed polymeric protectants. The inventors have discovered that in some variants, this increased heterogeneity can result in decreased activity of the polymer degradant. For instance, the presence of long and/or short polymeric protectants (particularly those with less than about half the average number molecular weight of the polymeric protectant and/or greater than about 150% of the average number molecular weight of the polymeric protectant) can measurably decrease the polymer degradant performance. To overcome this issue, the polymeric protectant formation process can include a size filtering process such as using chromatography (e.g., size exclusion chromatography, gel-permeation chromatography, etc.), filtration (e.g., tangential flow filtration, membrane filtration, dialysis, etc.), and/or other suitable processes to reduce the amount of one or both of small (e.g., the bottom 10% by mass, by polymer count, etc. of the polymeric protectants, the bottom 20% by mass, by polymer count, etc. of the polymeric protectants, etc.) and/or large (e.g., the top 10% by mass, by polymer count, etc. of the polymeric protectants, the top 20% by mass, by polymer count, etc. of the polymeric protectants, etc.) polymeric protectants that are formed. In some variations (in addition to or alternative to size filtering), the free radical polymerization can include stepwise introduction of monomers during the course of the polymerization (e.g., to control a distribution of free monomers at different times within the polymerization to more tightly control the resulting distribution of monomers within an uncontrolled free radical polymerization to more closely match a distribution of monomers within the polymeric nanoprotectant as formed via living polymerization). However, other suitable polymeric processes can be performed to improve the homogeneity (e.g., decrease dispersity in size, monomer distribution, etc.).

The polymerization reaction is typically initiated using an initiator. The initiator can be a thermal initiator (e.g., an initiator heated to a threshold temperature to activate the initiator or begin the polymerization reaction), a mechanical initiator (e.g., upon a threshold force application), electromagnetic initiator (e.g., by illumination with electromagnetic radiation with sufficient wavelength, frequency, intensity, etc. such as gamma radiation, x-rays, ultraviolet radiation, visible radiation, etc.), electrochemical initiator (e.g., activated at a threshold electrical potential), and/or can otherwise be activated. Examples of initiators include 1-1′-azobis(cyclohexanecarbonitrile), 2,2′-azobisisobutyronitrile (AIBN), 2,2-Bis(tert-butylperoxy) butane, 1,1-bis(tert-butylperoxy)cyclohexane, 2,2′-azobis [2-(2-imidazolin-2-yl)-propane]dihydrochloride, 2,5-bis(tert-butylperoxy)-2,5-dimethylhexane, 2,5-bis(tert-butylperoxy)-2,5-dimethyl-3-hexyne, bis(1-(tertbutylperoxy)-1-methylethyl)benzene, 1,1-bis(tert-butylperoxy)-3,3,5-trimethylcyclohexane, tert-butyl peracetate, tert-butyl hydroperoxide (TBHP), cumene hydroperoxide, di-tert-butyl peroxide, tert-butyl peroxybenzoate, tert-butylperoxyisopropyl carbonate, dicumyl peroxide, benzoyl peroxide, lauroyl peroxide, 2,4-pentanedione peroxide, peracetic acid, tert-amyl peroxybenzoate, 4,4-azobis(4-cyanovaleric acid), 1,1′-azobis(cyclohexanecarbonitrile) (ABCN or ACHN), ammonium persulfate, potassium persulfate (or other persulfate salts), lauroyl peroxide, tert-butyl peroxide, tert-butyl peroxybenzoate, benzoyl peroxide (BPO), phenylbis(2,4,6-trimethylbenzoyl) phosphine oxide), 2,2-dimethoxy-2-phenylacetophenone (DMPA), and/or any suitable initiator can be used. The concentration of initiator is typically between about 0.01% about 1.0% (e.g., wt % such as 0.01%, 0.02%, 0.03%, 0.05%, 0.1%, 0.2%, 0.3%, 0.5%, 0.7%, 0.75%, 0.9%, 1.0%, values or ranges therebetween, etc.) relative to a polymer precursor.

For the polymerization reaction, a total monomer concentration is typically between about 1 and 10 molar (where a concentration of an individual monomer can depend on the target composition of the final polymeric protectant). However, other suitable concentrations can be used. The polymerization reaction can be performed in ethanol, methanol, diethyl ether, dimethyl formamide (DMF), acetone, 2-butanol, methyl acetate, dioxane, dihydrolevoglucosenone, N-methyl-2-pyrrolidone (NMP), dimethyl sulfoxide, 2-pyrrolidone, 1,3-dimethyl-2-imidazolidinone, N,N′-dimethylpropyleneurea, hexamethylphosphoramide, dimethylfuran, tetrahydrofuran (THF), acetonitrile, ethyl acetate, pyridine, sulfolane, combinations thereof, and/or other solvent(s) can be used.

A number average molecular weight (e.g., measured based on colligative properties such as freezing point depression, end group analysis, ¹H NMR, gel permeation chromatography, etc.) or weight average molecular weight (e.g., measured using light scattering, gel permeation chromatography, etc.) of a polymeric protectant is typically between about 10 kD and 1000 kD (e.g., 20 kD, 50 kD, 100 kD, 200 kD, 500 kD, 1000 kD, values or ranges therebetween, etc.). However, the polymeric protectant can have a larger weight. A polydispersity index (D) of the polymeric protectant is preferably ≤2 (e.g., <1.8, <1.6, <1.5, <1.4, <1.2, 1.4-1.8, 1.4-1.6, 1.2-1.8, 1.1-1.4, 1.1-1.6, 1.6-1.8, other values or ranges therebetween). However, the polymeric substance can have other suitable Ð.

In variants, the nanoprotected polymer degradant can be included in the polymeric substance via solvent processing, additive processing (e.g., treating the nanoprotected polymer degradant as an additive), and/or using any suitable processing techniques (e.g., the nanoprotected polymer degradant can be used in the same processing method as used for the polymeric substance to prepare, shape, etc. the polymeric substance for an application). As a specific example, the nanoprotected polymer degradant can be mixed into the polymeric substance resin (e.g., where the resin is then used to form a material from the resin such as via molding, extrusion, thermoforming, compression molding, injection molding, high pressure lamination, etc.). However, the nanoprotected polymer degradant could be applied at the same time as the polymeric substance resin, applied on a surface of the polymeric substance resin (e.g., potentially with heat, pressure, force, etc. to influence diffusion, injection, etc. into the polymeric substance), and/or can otherwise be applied to form a material that includes the polymeric substance and the nanoprotected polymer degradant.

Typically, the polymer degradant will begin degrading the polymeric substance after one or more degradation condition(s) (e.g., trigger condition such as temperature, pressure, humidity, time, optical activation, chemical exposure, etc.) has occurred. However, additionally or alternatively, the nano-protectant can release the polymer degradant upon exposure to the degradation condition (e.g., where the polymer degradant can depolymerize the polymeric substance after release), and/or the polymeric degradant can otherwise degrade the polymeric substance (e.g., after use of the polymeric substance, after a threshold time, etc.).

### SPECIFIC EXAMPLES

A numbered list of specific examples of the technology described herein are provided below. A person of skill in the art will recognize that the scope of the technology is not limited to and/or by these specific examples.

1. A composition comprising:
a polymer selected from the group of polymers consisting of: polycarbonates, polyethers, polyetherketones, polyimides, polyamides, polyamide imides, polyether imides, polysulfones, polythiophenes, polyureas, polyurethanes, polysulfides, polyvinyl ethers, polyvinyl esters, polyvinyl alcohols, polyvinyl amines, polyvinyl amides, biopolymer, and blends thereof;
one or more enzymes each independently selected from the group of enzymes consisting of: oxidoreductases, transferases, hydrolases, lyases, isomerases, ligases, translocases, amidases, peptidases, proteases, proteinases, trypsins, subtilisins, hydrolases, lipases, phosphatases, polyurethanases, cellulases, RNAses, DNAses, peroxidases, laccases, urease, elastases, papains, oxidases, reductases, latex clearing proteins, cytochromes, alkane hydroxylases, alkane monoxygenases, dioxygenases, carboxylesterases, cutinases, alcohol reductases, hydroxylases, and combinations thereof; and
a polymeric nanoprotectant surrounding the enzyme, wherein the polymeric nanoprotectant comprises a first hydrophobic monomer, a charged monomer, and a polar monomer.

2. The composition of Specific Example 1, wherein the polymeric nanoprotectant further comprises a second hydrophobic monomer that is more hydrophobic than the first hydrophobic monomer.

3. The composition of Specific Example 2, wherein the second hydrophobic monomer has a hydrophilic-lipophilic balance less than 7.5.

4. The composition of any of Specific Examples 1-3, wherein the charged monomer comprises an anionic monomer at pH7.

5. The composition of Specific Example 4, wherein the anionic monomer is selected from the group consisting of: sodium acrylate, potassium acrylate, sulfopropyl acrylate, sulfoethyl acrylate, sulfomethyl acrylate, sulfo (2-methylpropyl) acrylate, acrylo t-butyl sulfonic acid, acrylate carboxylates, acrylate sulfates, acrylate phosphates, acrylate nitrates, sodium methacrylate, potassium methacrylate, sulfopropyl methacrylate, sulfoethyl methacrylate, sulfomethyl methacrylate, sulfo (2-methylpropyl) methacrylate, methacrylo t-butyl sulfonic acid, methacrylate carboxylates, methacrylate sulfates, methacrylate phosphates, methacrylate nitrates, and combinations thereof.

6. The composition of any of Specific Examples 1-5, wherein the charged monomer comprises a cationic monomer at pH7.

7. The composition of Specific Example 6, wherein the cationic monomer is selected from the group consisting of: 2-aminomethyl acrylate, 2-aminoethyl acrylate, 3-aminopropyl acrylate, N-[3-(dimethylamino) propyl]-2-acrylate, 3-acrylopropyl trimethylammonium salt, acryloyl oxyethyl dimethylbenzyl ammonium salt, acryloyl oxyethyl dimethyl ethyl ammonium salt, acryloyl oxyethyl trimethyl ammonium salt, acryloyl oxyethyl triethyl ammonium, 2-aminomethyl methacrylate, 2-aminoethyl methacrylate, 3-aminopropyl methacrylate, N-[3-(dimethylamino) propyl]-2-methacrylate, 3-methacrylopropyl trimethylammonium salt, methacryloyl oxyethyl dimethylbenzyl ammonium salt, methacryloyl oxyethyl dimethyl ethyl ammonium salt, methacryloyl oxyethyl trimethyl ammonium salt, methacryloyl oxyethyl triethyl ammonium, and combinations thereof.

8. The composition of any of Specific Examples 1 or 4-7, wherein the first hydrophobic monomer is selected from the group consisting of: methyl acrylate, ethyl acrylate, n-propyl acrylate, i-propyl acrylate, n-butyl acrylate, i-butyl acrylate, t-butyl acrylate, pentyl acrylate, hexyl acrylate, heptyl acrylate, octyl acrylate, nonyl acrylate, decyl acrylate, 2-ethylhexyl acrylate, lauryl acrylate, stearyl acrylate, isobornyl acrylate, styrene, methyl methacrylate, ethyl methacrylate, n-propyl methacrylate, i-propyl methacrylate, n-butyl methacrylate, i-butyl methacrylate, t-butyl methacrylate, pentyl methacrylate, hexyl methacrylate, heptyl methacrylate, octyl methacrylate, nonyl methacrylate, decyl methacrylate, 2-ethylhexyl methacrylate, lauryl methacrylate, stearyl methacrylate, isobornyl methacrylate, α-methylstyrene, and combinations thereof.

9. The composition of any of specific examples 1-8, wherein the polar monomer is selected from the group consisting of: ethoxy diethylene glycol acrylate, methoxy triethylene glycol acrylate, methoxy dipropylene glycol acrylate, phenoxyethyl acrylate, phenoxydiethylene glycol acrylate, phenoxy polyethylene glycol acrylate, m-phenoxybenzyl acrylate, 2-hydroxyl butyl acrylate, 2-hydroxy-3-phenoxypropyl acrylate, 2-acryloloxyethyl succinate, 2-acryloyloxyethyl hexahydro phthalate, 2-acryloyloxyethyl phthalate, 2-acryloyloxyethyl 2-hydroxyethyl phthalate, triethylene glycol diacrylate, tetrahydrobenzyl acrylate, hexahydrobenzyl acrylate, hydroxyethyl acrylate, acrylate polyethylene glycol (PEG) oligomer with a PEG molecular weight between 100 Da and 2000 Da, acrylate polypropylene glycol (PPG) oligomer with a PPG molecular weight between 100 Da and 2000 Da, acrylate polybutylene glycol (PBG) oligomer with a PBG molecular weight between 100 Da and 2000 Da, ethoxy diethylene glycol methacrylate, methoxy triethylene glycol methacrylate, methoxy dipropylene glycol methacrylate, phenoxyethyl methacrylate, phenoxydiethylene glycol methacrylate, phenoxy polyethylene glycol methacrylate, m-phenoxybenzyl methacrylate, 2-hydroxyl butyl methacrylate, 2-hydroxy-3-phenoxypropyl methacrylate, 2-methacryloloxyethyl succinate, 2-methacryloyloxyethyl hexahydro phthalate, 2-methacryloyloxyethyl phthalate, 2-methacryloyloxyethyl 2-hydroxyethyl phthalate, triethylene glycol tetrahydrobenzyl methacrylate, hexahydrobenzyl methacrylate, hydroxyethyl methacrylate, methacrylate polyethylene glycol (PEG) oligomer with a PEG molecular weight between 100 Da and 2000 Da, methacrylate polypropylene glycol (PPG) oligomer with a PPG molecular weight between 100 Da and 2000 Da, methacrylate polybutylene glycol (PBG) oligomer with a PBG molecular weight between 100 Da and 2000 Da, and combinations thereof.

10. The composition of any of specific examples 1-9, wherein the enzyme is selected to progressively degrade the polymer.

11. The composition of any of Specific Examples 1-9, wherein the enzyme is selected to degrade the polymer via random chain scission.

12. The composition of any of specific examples 1-11, wherein the enzyme and the polymeric nanoprotectant form a cluster, wherein the enzyme is embedded within the polymeric nanoprotectant.

13. The composition of Specific Example 12, wherein a size of the cluster is between 1-500 nm.

14. A method comprising:
forming a mixture comprising a first hydrophobic monomer, a charged monomer comprising a positive or negative net charge at pH 7, a polar monomer comprising no net charge at pH 7, and an initiator;
activating the initiator to begin polymerization of the first hydrophobic monomer, the charger monomer, and the polar monomer via radical polymerization to form a polymeric protectant; and
purifying the polymeric protectants resulting from said polymerization.

15 The method of Specific Example 14, wherein purifying the polymeric protectants comprises filtering the polymeric protectants using filtration, chromatography, dialysis, or tangential flow filtration based on a molecular size to remove polymeric protectants with molecular mass less than a threshold mass.

16. The method of any of Specific Examples 14-15, wherein purifying the polymeric protectants comprises filtering the polymeric protectants using filtration, chromatography, dialysis, or tangential flow filtration based on a molecular size to remove polymeric protectants with molecular mass greater than a threshold mass.

17. The method of any of Specific Examples 14-16, further comprising, during the polymerization of the first hydrophobic monomer, the charger monomer, and the polar monomer, adding additional of the first hydrophobic monomer, the charger monomer, and the polar monomer to control a concentration of each monomer within the mixture.

18. The method of Specific Example 17, further comprising mixing the purified polymeric protectants with an enzyme to form a nanoprotected enzyme complex.

19 The method of Specific Example 18, further comprising incorporating the nanoprotected enzyme complex into a polymer resin, wherein the enzyme progressively depolymerizes the polymer formed from the polymer resin when target conditions are met.

20. The method of Specific Example 18, further comprising incorporating the nanoprotected enzyme complex into a polymer resin, wherein the enzyme depolymerizes the polymer formed from the polymer resin through random chain scission when target conditions are met.

21. The method of any of Specific Examples 14-20, wherein the mixture further comprises a second hydrophobic monomer comprising a hydrophilic-lipophilic balance less than 7.5, wherein the first hydrophobic monomer comprises a hydrophilic-lipophilic balance greater than or equal to 7.5.

22. The method of any of Specific Examples 14-20, wherein:
the first hydrophobic monomer is selected from the group consisting of: methyl acrylate, ethyl acrylate, n-propyl acrylate, i-propyl acrylate, methyl methacrylate, ethyl methacrylate, n-propyl methacrylate, i-propyl methacrylate, n-butyl acrylate, i-butyl acrylate, t-butyl acrylate, pentyl acrylate, hexyl acrylate, heptyl acrylate, octyl acrylate, nonyl acrylate, decyl acrylate, 2-ethylhexyl acrylate, lauryl acrylate, stearyl acrylate, isobornyl acrylate, styrene, n-butyl methacrylate, i-butyl methacrylate, t-butyl methacrylate, pentyl methacrylate, hexyl methacrylate, heptyl methacrylate, octyl methacrylate, nonyl methacrylate, decyl methacrylate, 2-ethylhexyl methacrylate, lauryl methacrylate, stearyl methacrylate, isobornyl methacrylate, α-methylstyrene, and combinations thereof;
the charged monomer is selected from the group consisting of: 2-aminoethyl acrylate, 3-aminopropyl acrylate, N-[3-(dimethylamino) propyl]-2-acrylate, 3-acrylopropyl trimethylammonium salt, acryloyl oxyethyl dimethylbenzyl ammonium salt, acryloyl oxyethyl dimethyl ethyl ammonium salt, acryloyl oxyethyl trimethyl ammonium salt, acryloyl oxyethyl triethyl ammonium, 2-aminoethyl methacrylate, 3-aminopropyl methacrylate, N-[3-(dimethylamino) propyl]-2-methacrylate, 3-methacrylopropyl trimethylammonium salt, methacryloyl oxyethyl dimethylbenzyl ammonium salt, methacryloyl oxyethyl dimethyl ethyl ammonium salt, methacryloyl oxyethyl trimethyl ammonium salt, methacryloyl oxyethyl triethyl ammonium, sodium acrylate, potassium acrylate, sulfopropyl acrylate, sulfoethyl acrylate, sulfomethyl acrylate, sulfo (2-methylpropyl) acrylate, acrylo t-butyl sulfonic acid, acrylate carboxylates, acrylate sulfates, acrylate phosphates, acrylate nitrates, sodium methacrylate, potassium methacrylate, sulfopropyl methacrylate, sulfoethyl methacrylate, sulfomethyl methacrylate, sulfo (2-methylpropyl) methacrylate, methacrylo t-butyl sulfonic acid, methacrylate carboxylates, methacrylate sulfates, methacrylate phosphates, methacrylate nitrates, and combinations thereof; and
the polar monomer is selected from the group consisting of: ethoxy diethylene glycol acrylate, methoxy triethylene glycol acrylate, methoxy dipropylene glycol acrylate, phenoxyethyl acrylate, phenoxydiethylene glycol acrylate, phenoxy polyethylene glycol acrylate, m-phenoxybenzyl acrylate, 2-hydroxyl butyl acrylate, 2-hydroxy-3-phenoxypropyl acrylate, 2-acryloloxyethyl succinate, 2-acryloyloxyethyl hexahydro phthalate, 2-acryloyloxyethyl phthalate, 2-acryloyloxyethyl 2-hydroxyethyl phthalate, triethylene glycol diacrylate, tetrahydrobenzyl acrylate, hexahydrobenzyl acrylate, hydroxyethyl acrylate, acrylate polyethylene glycol (PEG) oligomer with a PEG molecular weight between 100 Da and 2000 Da, acrylate polypropylene glycol (PPG) oligomer with a PPG molecular weight between 100 Da and 2000 Da, acrylate polybutylene glycol (PBG) oligomer with a PBG molecular weight between 100 Da and 2000 Da, ethoxy diethylene glycol methacrylate, methoxy triethylene glycol methacrylate, methoxy dipropylene glycol methacrylate, phenoxyethyl methacrylate, phenoxydiethylene glycol methacrylate, phenoxy polyethylene glycol methacrylate, m-phenoxybenzyl methacrylate, 2-hydroxyl butyl methacrylate, 2-hydroxy-3-phenoxypropyl methacrylate, 2-methacryloloxyethyl succinate, 2-methacryloyloxyethyl hexahydro phthalate, 2-methacryloyloxyethyl phthalate, 2-methacryloyloxyethyl 2-hydroxyethyl phthalate, triethylene glycol tetrahydrobenzyl methacrylate, hexahydrobenzyl methacrylate, hydroxyethyl methacrylate, methacrylate polyethylene glycol (PEG) oligomer with a PEG molecular weight between 100 Da and 2000 Da, methacrylate polypropylene glycol (PPG) oligomer with a PPG molecular weight between 100 Da and 2000 Da, methacrylate polybutylene glycol (PBG) oligomer with a PBG molecular weight between 100 Da and 2000 Da, and combinations thereof.

23. The polymeric nanoprotectant of any of specific examples 1-13.

24. The polymeric nanoprotectant as formed by the method of any of specific examples 14-22.

25. A method for decomposing a polymeric substance or polymeric resin using the composition of any of Specific Examples 1-13.

26. A method for forming a polymeric substance incorporating the enzymes and polymeric nanoprotectant of any of specific examples 1-13.

Embodiments of the composition and/or method can include every combination and permutation of the various composition constituents and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the composition, elements, and/or entities described herein. Components and/or processes of the preceding composition and/or method can be used with, in addition to, in lieu of, or otherwise integrated with all or a portion of the composition and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A composition comprising:
a polymer selected from the group of polymers consisting of: polycarbonates, polyethers, polyetherketones, polyimides, polyamides, polyamide imides, polyether imides, polysulfones, polythiophenes, polyureas, polyurethanes, polysulfides, polyvinyl ethers, polyvinyl esters, polyvinyl alcohols, polyvinyl amines, polyvinyl amides, biopolymer, and blends thereof;
one or more enzymes each independently selected from the group of enzymes consisting of: oxidoreductases, transferases, hydrolases, lyases, isomerases, ligases, translocases, amidases, peptidases, proteases, proteinases, trypsins, subtilisins, hydrolases, lipases, phosphatases, polyurethanases, cellulases, RNAses, DNAses, peroxidases, laccases, urease, elastases, papains, oxidases, reductases, latex clearing proteins, cytochromes, alkane hydroxylases, alkane monoxygenases, dioxygenases, carboxylesterases, cutinases, alcohol reductases, hydroxylases, and combinations thereof; and
a polymeric nanoprotectant surrounding the enzyme, wherein the polymeric nanoprotectant comprises a first hydrophobic monomer, a charged monomer, and a polar monomer.

2. The composition of claim 1, wherein the polymeric nanoprotectant further comprises a second hydrophobic monomer that is more hydrophobic than the first hydrophobic monomer.

3. The composition of claim 2, wherein the second hydrophobic monomer has a hydrophilic-lipophilic balance less than 7.5.

4. The composition of claim 1, wherein the charged monomer comprises an anionic monomer at pH7.

5. The composition of claim 4, wherein the anionic monomer is selected from the group consisting of: sodium acrylate, potassium acrylate, sulfopropyl acrylate, sulfoethyl acrylate, sulfomethyl acrylate, sulfo (2-methylpropyl) acrylate, acrylo t-butyl sulfonic acid, acrylate carboxylates, acrylate sulfates, acrylate phosphates, acrylate nitrates, sodium methacrylate, potassium methacrylate, sulfopropyl methacrylate, sulfoethyl methacrylate, sulfomethyl methacrylate, sulfo (2-methylpropyl) methacrylate, methacrylo t-butyl sulfonic acid, methacrylate carboxylates, methacrylate sulfates, methacrylate phosphates, methacrylate nitrates, and combinations thereof.

6. The composition of claim 1, wherein the charged monomer comprises a cationic monomer at pH7.

7. The composition of claim 6, wherein the cationic monomer is selected from the group consisting of: 2-aminomethyl acrylate, 2-aminoethyl acrylate, 3-aminopropyl acrylate, N-[3-(dimethylamino) propyl]-2-acrylate, 3-acrylopropyl trimethylammonium salt, acryloyl oxyethyl dimethylbenzyl ammonium salt, acryloyl oxyethyl dimethyl ethyl ammonium salt, acryloyl oxyethyl trimethyl ammonium salt, acryloyl oxyethyl triethyl ammonium, 2-aminomethyl methacrylate, 2-aminoethyl methacrylate, 3-aminopropyl methacrylate, N-[3-(dimethylamino) propyl]-2-methacrylate, 3-methacrylopropyl trimethylammonium salt, methacryloyl oxyethyl dimethylbenzyl ammonium salt, methacryloyl oxyethyl dimethyl ethyl ammonium salt, methacryloyl oxyethyl trimethyl ammonium salt, methacryloyl oxyethyl triethyl ammonium, and combinations thereof.

8. The composition of claim 1, wherein the first hydrophobic monomer is selected from the group consisting of: methyl acrylate, ethyl acrylate, n-propyl acrylate, i-propyl acrylate, n-butyl acrylate, i-butyl acrylate, t-butyl acrylate, pentyl acrylate, hexyl acrylate, heptyl acrylate, octyl acrylate, nonyl acrylate, decyl acrylate, 2-ethylhexyl acrylate, lauryl acrylate, stearyl acrylate, isobornyl acrylate, styrene, methyl methacrylate, ethyl methacrylate, n-propyl methacrylate, i-propyl methacrylate, n-butyl methacrylate, i-butyl methacrylate, t-butyl methacrylate, pentyl methacrylate, hexyl methacrylate, heptyl methacrylate, octyl methacrylate, nonyl methacrylate, decyl methacrylate, 2-ethylhexyl methacrylate, lauryl methacrylate, stearyl methacrylate, isobornyl methacrylate, α-methylstyrene, and combinations thereof.

9. The composition of claim 1, wherein the polar monomer is selected from the group consisting of: ethoxy diethylene glycol acrylate, methoxy triethylene glycol acrylate, methoxy dipropylene glycol acrylate, phenoxyethyl acrylate, phenoxydiethylene glycol acrylate, phenoxy polyethylene glycol acrylate, m-phenoxybenzyl acrylate, 2-hydroxyl butyl acrylate, 2-hydroxy-3-phenoxypropyl acrylate, 2-acryloloxyethyl succinate, 2-acryloyloxyethyl hexahydro phthalate, 2-acryloyloxyethyl phthalate, 2-acryloyloxyethyl 2-hydroxyethyl phthalate, triethylene glycol diacrylate, tetrahydrobenzyl acrylate, hexahydrobenzyl acrylate, hydroxyethyl acrylate, acrylate polyethylene glycol (PEG) oligomer with a PEG molecular weight between 100 Da and 2000 Da, acrylate polypropylene glycol (PPG) oligomer with a PPG molecular weight between 100 Da and 2000 Da, acrylate polybutylene glycol (PBG) oligomer with a PBG molecular weight between 100 Da and 2000 Da, ethoxy diethylene glycol methacrylate, methoxy triethylene glycol methacrylate, methoxy dipropylene glycol methacrylate, phenoxyethyl methacrylate, phenoxydiethylene glycol methacrylate, phenoxy polyethylene glycol methacrylate, m-phenoxybenzyl methacrylate, 2-hydroxyl butyl methacrylate, 2-hydroxy-3-phenoxypropyl methacrylate, 2-methacryloloxyethyl succinate, 2-methacryloyloxyethyl hexahydro phthalate, 2-methacryloyloxyethyl phthalate, 2-methacryloyloxyethyl 2-hydroxyethyl phthalate, triethylene glycol tetrahydrobenzyl methacrylate, hexahydrobenzyl methacrylate, hydroxyethyl methacrylate, methacrylate polyethylene glycol (PEG) oligomer with a PEG molecular weight between 100 Da and 2000 Da, methacrylate polypropylene glycol (PPG) oligomer with a PPG molecular weight between 100 Da and 2000 Da, methacrylate polybutylene glycol (PBG) oligomer with a PBG molecular weight between 100 Da and 2000 Da, and combinations thereof.

10. The composition of claim 1, wherein the enzyme is selected to progressively degrade the polymer.

11. The composition of claim 1, wherein the enzyme is selected to degrade the polymer via random chain scission.

12. The composition of claim 1, wherein the enzyme and the polymeric nanoprotectant form a cluster, wherein the enzyme is embedded within the polymeric nanoprotectant.

13. The composition of claim 12, wherein a size of the cluster is between 1-500 nm.

14. A method comprising:
forming a mixture comprising a first hydrophobic monomer, a charged monomer comprising a positive or negative net charge at pH 7, a polar monomer comprising no net charge at pH 7, and an initiator;
activating the initiator to begin polymerization of the first hydrophobic monomer, the charger monomer, and the polar monomer via radical polymerization to form a polymeric protectant; and
purifying the polymeric protectants resulting from said polymerization.

15. The method of claim 14, wherein purifying the polymeric protectants comprises filtering the polymeric protectants using filtration, chromatography, dialysis, or tangential flow filtration based on a molecular size to remove polymeric protectants with molecular mass less than a threshold mass.

16. The method of claim 14, wherein purifying the polymeric protectants comprises filtering the polymeric protectants using filtration, chromatography, dialysis, or tangential flow filtration based on a molecular size to remove polymeric protectants with molecular mass greater than a threshold mass.

17. The method of claim 14, further comprising, during the polymerization of the first hydrophobic monomer, the charger monomer, and the polar monomer, adding additional of the first hydrophobic monomer, the charger monomer, and the polar monomer to control a concentration of each monomer within the mixture.

18. The method of claim 17, further comprising mixing the purified polymeric protectants with an enzyme to form a nanoprotected enzyme complex.

19. The method of claim 18, further comprising incorporating the nanoprotected enzyme complex into a polymer resin, wherein the enzyme progressively depolymerizes the polymer formed from the polymer resin when target conditions are met.

20. The method of claim 18, further comprising incorporating the nanoprotected enzyme complex into a polymer resin, wherein the enzyme depolymerizes the polymer formed from the polymer resin through random chain scission when target conditions are met.

21. The method of claim 18, wherein the mixture further comprises a second hydrophobic monomer comprising a hydrophilic-lipophilic balance less than 7.5, wherein the first hydrophobic monomer comprises a hydrophilic-lipophilic balance greater than or equal to 7.5.

22. The method of claim 18, wherein:
the first hydrophobic monomer is selected from the group consisting of: methyl acrylate, ethyl acrylate, n-propyl acrylate, i-propyl acrylate, methyl methacrylate, ethyl methacrylate, n-propyl methacrylate, i-propyl methacrylate, n-butyl acrylate, i-butyl acrylate, t-butyl acrylate, pentyl acrylate, hexyl acrylate, heptyl acrylate, octyl acrylate, nonyl acrylate, decyl acrylate, 2-ethylhexyl acrylate, lauryl acrylate, stearyl acrylate, isobornyl acrylate, styrene, n-butyl methacrylate, i-butyl methacrylate, t-butyl methacrylate, pentyl methacrylate, hexyl methacrylate, heptyl methacrylate, octyl methacrylate, nonyl methacrylate, decyl methacrylate, 2-ethylhexyl methacrylate, lauryl methacrylate, stearyl methacrylate, isobornyl methacrylate, α-methylstyrene, and combinations thereof;
the charged monomer is selected from the group consisting of: 2-aminoethyl acrylate, 3-aminopropyl acrylate, N-[3-(dimethylamino) propyl]-2-acrylate, 3-acrylopropyl trimethylammonium salt, acryloyl oxyethyl dimethylbenzyl ammonium salt, acryloyl oxyethyl dimethyl ethyl ammonium salt, acryloyl oxyethyl trimethyl ammonium salt, acryloyl oxyethyl triethyl ammonium, 2-aminoethyl methacrylate, 3-aminopropyl methacrylate, N-[3-(dimethylamino) propyl]-2-methacrylate, 3-methacrylopropyl trimethylammonium salt, methacryloyl oxyethyl dimethylbenzyl ammonium salt, methacryloyl oxyethyl dimethyl ethyl ammonium salt, methacryloyl oxyethyl trimethyl ammonium salt, methacryloyl oxyethyl triethyl ammonium, sodium acrylate, potassium acrylate, sulfopropyl acrylate, sulfoethyl acrylate, sulfomethyl acrylate, sulfo (2-methylpropyl) acrylate, acrylo t-butyl sulfonic acid, acrylate carboxylates, acrylate sulfates, acrylate phosphates, acrylate nitrates, sodium methacrylate, potassium methacrylate, sulfopropyl methacrylate, sulfoethyl methacrylate, sulfomethyl methacrylate, sulfo (2-methylpropyl) methacrylate, methacrylo t-butyl sulfonic acid, methacrylate carboxylates, methacrylate sulfates, methacrylate phosphates, methacrylate nitrates, and combinations thereof; and
the polar monomer is selected from the group consisting of: ethoxy diethylene glycol acrylate, methoxy triethylene glycol acrylate, methoxy dipropylene glycol acrylate, phenoxyethyl acrylate, phenoxydiethylene glycol acrylate, phenoxy polyethylene glycol acrylate, m-phenoxybenzyl acrylate, 2-hydroxyl butyl acrylate, 2-hydroxy-3-phenoxypropyl acrylate, 2-acryloloxyethyl succinate, 2-acryloyloxyethyl hexahydro phthalate, 2-acryloyloxyethyl phthalate, 2-acryloyloxyethyl 2-hydroxyethyl phthalate, triethylene glycol diacrylate, tetrahydrobenzyl acrylate, hexahydrobenzyl acrylate, hydroxyethyl acrylate, acrylate polyethylene glycol (PEG) oligomer with a PEG molecular weight between 100 Da and 2000 Da, acrylate polypropylene glycol (PPG) oligomer with a PPG molecular weight between 100 Da and 2000 Da, acrylate polybutylene glycol (PBG) oligomer with a PBG molecular weight between 100 Da and 2000 Da, ethoxy diethylene glycol methacrylate, methoxy triethylene glycol methacrylate, methoxy dipropylene glycol methacrylate, phenoxyethyl methacrylate, phenoxydiethylene glycol methacrylate, phenoxy polyethylene glycol methacrylate, m-phenoxybenzyl methacrylate, 2-hydroxyl butyl methacrylate, 2-hydroxy-3-phenoxypropyl methacrylate, 2-methacryloloxyethyl succinate, 2-methacryloyloxyethyl hexahydro phthalate, 2-methacryloyloxyethyl phthalate, 2-methacryloyloxyethyl 2-hydroxyethyl phthalate, triethylene glycol tetrahydrobenzyl methacrylate, hexahydrobenzyl methacrylate, hydroxyethyl methacrylate, methacrylate polyethylene glycol (PEG) oligomer with a PEG molecular weight between 100 Da and 2000 Da, methacrylate polypropylene glycol (PPG) oligomer with a PPG molecular weight between 100 Da and 2000 Da, methacrylate polybutylene glycol (PBG) oligomer with a PBG molecular weight between 100 Da and 2000 Da, and combinations thereof.
